(19) United States
(12) Patent Application Publication
(10) Pub. No.: US 2026/0162777 A1
(43) Pub. Date: Jun. 11, 2026

(54) METHOD FOR PREDICITING COMPOUND-PROTEIN BINDING AFFINITY AND APPARATUS THEREOF

(71) Applicant: UIF (University Industry Foundation), Yonsei University, Seoul (KR)

(72) Inventors: SANGHYUN PARK, Seoul (KR); SANGMIN SEO, Jeju-si (KR); SEUNGYEON CHOI, Seoul (KR); HWANHEE KIM, Seoul (KR); JIEUN LEE, Seoul (KR)

(21) Appl. No.: 19/411,216

(22) Filed: Dec. 6, 2025

(30) Foreign Application Priority Data
Dec. 6, 2024 (KR) 10-2024-0180165

(51) Int. Cl. G16C 20/30; G16C 20/80

(52) U.S. Cl. CPC G16C 20/30; G16C 20/80

(57) ABSTRACT

A compound-protein binding affinity prediction method performed by at least one processor includes receiving compound data and protein data, which interact with each other, generating an attribute vector of a compound and an attribute vector of a protein based on the input compound data and the input protein data, calculating an attention value based on the attribute vector of the compound and the attribute vector of the protein, generating a first interaction matrix based on the attention value, learning a first AI model to predict a binding affinity and a non-covalent interaction of compound-protein by using the first interaction matrix as learning data, and predicting the binding affinity and the non-covalent interaction of the compound-protein based on an output value of the first AI model.

### CROSS-REFERENCE TO RELATED APPLICATIONS

A claim for priority under 35 U.S.C. § 119 is made to Korean Patent Application No. 10-2024-0180165 filed on Nov. 6, 2024, in the Korean Intellectual Property Office, the entire contents of which are hereby incorporated by reference.

### BACKGROUND

Embodiments of the inventive concept described herein relate to a method for predicting compound-protein binding affinity, and an apparatus thereof, and more particularly, relate to a compound-protein binding affinity prediction method that accurately and effectively predicts compound-protein binding affinity based on compound-protein complex free.

High-throughput screening is used in the early stages of drug development, but it is impossible to evaluate all compound-protein interactions. To compensate for this, protein structure-based virtual screening is employed. For example, molecular docking is widely utilized. However, it has limitations in requiring significant computational resources and having restricted accuracy.

Moreover, the scarcity of experimentally obtained 3D compound-protein complex structures has limited training datasets, thereby hindering development.

Accordingly, inventors of the inventive concept endeavored to predict compound-protein binding affinity independently of compound-protein complex structures, thereby culminating in the completion of the inventive concept.

### SUMMARY

Embodiments of the inventive concept provide a compound-protein binding affinity prediction method that accurately and effectively predicts compound-protein binding affinity based on compound-protein complex free.

Problems to be solved by the inventive concept are not limited to the problems mentioned above, and other problems not mentioned will be clearly understood by those skilled in the art from the following description.

According to an exemplary embodiment, a compound-protein binding affinity prediction method performed by at least one processor includes receiving compound data and protein data, which interact with each other, generating an attribute vector of a compound and an attribute vector of a protein based on the input compound data and the input protein data, calculating an attention value based on the attribute vector of the compound and the attribute vector of the protein, generating a first interaction matrix based on the attention value, learning a first AI model to predict a binding affinity and a non-covalent interaction of compound-protein by using the first interaction matrix as learning data, and predicting the binding affinity and the non-covalent interaction of the compound-protein based on an output value of the first AI model.

According to an embodiment of the inventive concept, the receiving of the compound data includes generating a compound structure graph. The compound structure graph has an attribute of an atom as a node and has an attribute of a bond as an edge.

According to an embodiment of the inventive concept, the calculating of the attention value based on the attribute vector of the compound and the attribute vector of the protein includes calculating a key, a value, and a query based on the attribute vector of the compound and the attribute vector of the protein, and crossing the calculated query of the compound and the calculated query of the protein, or crossing a key and a value of the compound and a key and a value of the protein to provide the crossed result to a sub-attention layer.

According to an embodiment of the inventive concept, the calculating of the attention value based on the attribute vector of the compound and the attribute vector of the protein further includes providing an output value of the sub-attention layer to a self-attention layer, and providing an output value of the self-attention layer to a feed-forward layer.

According to an embodiment of the inventive concept, the learning of the first AI model includes calculating an interaction score between one or more elements of the compound and one or more residues of the protein, extracting a latent variable from the score, and providing the latent variable to a fully connected layer.

According to an embodiment of the inventive concept, the output value of the first AI model includes the attribute vector of the compound, the attribute vector of the protein, the first interaction matrix, and the binding affinity predicted by the first AI model.

According to an embodiment of the inventive concept, the predicting of the binding affinity and the non-covalent interaction of the compound-protein based on the output value of the first AI model includes performing knowledge distillation by inputting the output value of the first AI model into a second AI model, and predicting the non-covalent interaction and the binding affinity in the second AI model. Data received by the first AI model is based on a compound-protein complex.

According to an embodiment of the inventive concept, the performing of the knowledge distillation includes calculating a loss function by comparing the first interaction matrix with a second interaction matrix generated by the second AI model.

According to an embodiment of the inventive concept, a computer program stored in a computer-readable recording medium is provided to execute the above-described method on a computer.

According to an exemplary embodiment, a computing apparatus includes a communication module, a memory, and at least one processor connected to the memory and configured to execute at least one computer-readable program included in the memory. The at least one program includes instructions for receiving a structure of a compound and a structure of a protein, which interact with each other, generating an attribute vector of a compound and an attribute vector of a protein based on the input structure of the compound and the input structure of the protein, calculating an attention value based on the attribute vector of the compound and the attribute vector of the protein, generating a first interaction matrix based on the attention value, learning a first AI model to predict a binding affinity and a non-covalent interaction of compound-protein by using the first interaction matrix as learning data, and predicting the binding affinity and the non-covalent interaction of the compound-protein based on an output value of the first AI model.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a functional block diagram illustrating an internal configuration of a computing apparatus, according to an embodiment;

FIG. 2 is a functional block diagram showing an internal configuration of an AI model, according to an embodiment;

FIG. 3 is a flowchart of a compound-protein binding affinity prediction method, according to an embodiment;

FIG. 4 is a diagram illustrating an operation of calculating an attention value by using a layer, according to an embodiment;

FIG. 5 is a graph for comparing the performance of models predicting a binding affinity of compound-protein, according to an embodiment; and

FIG. 6 is a block diagram showing a hardware configuration of a computing apparatus, according to an embodiment.

### DETAILED DESCRIPTION

Hereinafter, embodiments of the inventive concept will be described in detail with reference to the accompanying drawings. However, the inventive concept is not intended to be limited or restricted by embodiments. Unless otherwise defined, all terms (including technical and scientific terms) used in the specification should have the same meaning as commonly understood by those skilled in the art to which the inventive concept pertains, but which may vary according to the intent or precedent of those practicing in the art, the emergence of new technology, and the like.

Moreover, terms, such as those defined in commonly used dictionaries, should not be interpreted in an idealized or overly formal sense unless expressly so defined herein. Terms arbitrarily selected by the applicant of embodiments may also be used in a specific case. In this case, the detailed meanings are given in the corresponding description. Hence, these terms used in the inventive concept may be defined based on their meanings and the contents of the inventive concept, not by simply stating the terms.

It will be understood that the terms “comprises,” “comprising,” “includes” and/or “including,” when used herein, specify the presence of stated elements and/or components, but do not preclude the presence or addition of one or more other elements and/or components. Moreover, as used in the specification, the singular terms “a,” “an” and “the” are intended to include the plural forms as well, unless the context clearly indicates otherwise. Besides, the expression “at least one of a, b, and/or c” described throughout this specification may encompass ‘a alone’, ‘b alone’, ‘c alone’, ‘a and b’, ‘a and c’, ‘b and c’, or ‘all of a, b, and c’.

In the meantime, the term “first and/or second” used in the specification will be used to describe various elements but will be described only for the purpose of distinguishing one element from another element, not limiting an element of the corresponding term. For example, without departing the scope of the inventive concept, a first element may be referred to as a second element, and similarly, a second element may be referred to as a first element.

In addition, terms such as “ . . . unit”, “ . . . module”, etc. described in this specification mean a unit that processes at least one function or operation, which may be implemented as hardware or software, or a combination of hardware and software. Furthermore, an embodiment of the inventive concept described herein may be represented by functional block configurations and various processing steps. These functional blocks may be implemented in the variable number of hardware or/and software configurations that perform specific functions. For example, embodiments of the inventive concept may employ integrated circuit configurations such as a memory, processing, logic, a look-up table, etc., which may execute various functions under the control of one or more microprocessors or other control devices.

In an embodiment of the inventive concept, functions related to artificial intelligence may be implemented through a processor and a memory. In this case, the processor may be one of a general-purpose processor such as a center processing unit (CPU), an application processor (AP), a digital signal processor (DSP), a graphics-dedicated processor such as a graphic processing unit (GPU), a vision processing unit (VPU), and an AI-dedicated processor such as a neural network processing unit (NPU). The processor may process input data depending on an AI model or a predefined operating rule, which is stored in the memory. Alternatively, when the processor is an AI-dedicated processor, the AI-dedicated processor may be designed with a hardware structure specialized for the processing of a specific AI model. In some embodiments of the inventive concept, functions related to artificial intelligence may be implemented through a plurality of processors.

In an embodiment of the inventive concept, the predefined operating rule or the artificial intelligence model may be configured to perform machine learning. Here, being configured to perform machine learning means that the predefined operation rule or the artificial intelligence model is configured to perform a desired feature (or purpose) by learning pieces of learning data based on a learning algorithm. This learning may be performed by a device itself, on which the artificial intelligence according to an embodiment of the inventive concept is implemented, or may be performed through a separate server and/or system.

The artificial intelligence model may be implemented with a neural network (or an artificial neural network) and may operate based on a statistical learning algorithm that mimics biological neurons in machine learning and cognitive science. The neural network may refer to a model as a whole having the ability to solve problems as artificial neurons (nodes), which form a network by connecting synapses, changes the strength of their synaptic connections through learning. The neural network may be composed of a plurality of neural network layers. For example, the neural network may include an input layer, a hidden layer, and an output layer. Each of the plurality of neural network layers may include at least one node and at least one weight, and may perform neural network operations through operations between weights and the operation results of the previous layer. At least one weight of the plurality neural network layers may be optimized by the training result of the artificial intelligence model. For example, during the training process, the at least one weight may be updated such that a loss value or cost value obtained from the artificial intelligence model is reduced or minimized. The neural network may infer the desired result from an arbitrary input.

Training methods of the artificial intelligence model may be classified into supervised learning, in which input data and output data are provided as training data according to the learning method, and the correct answer (output data) corresponding to the problem (input data) is determined, unsupervised learning, in which only the input data is provided without the output data, and the correct answer (output data) corresponding to the problem (input data) is not determined, reinforcement learning, in which a reward is given whenever an action is taken in a current state, and training proceeds to maximize the reward, and the like. Alternatively, the training methods may be distinguished based on the architecture, which is the structure of the learning model.

According to an embodiment of the inventive concept, the artificial intelligence model may use at least one of various artificial intelligence structures and algorithms such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, a classification network, Generative Modeling, explainable AI, Continual AI, Representation Learning, AI for Material Design, algorithms for natural language processing (e.g., BERT, SP-BERT, MRC/QA, Text Analysis, Dialog System, GPT-3, and GPT-4), algorithms for vision processing (e.g., Visual Analytics, Visual Understanding, Video Synthesis, and ResNet), algorithms for data intelligence (e.g., Anomaly Detection, Prediction, Time-Series Forecasting, Optimization, Recommendation, and Data Creation), but is not limited thereto. The above-described examples are merely illustrative of artificial intelligence structures and algorithms used in accordance with embodiments of the inventive concept, and do not limit the artificial intelligence structures and algorithms used in accordance with embodiments of the inventive concept.

Hereinafter, various embodiments of the inventive concept will be described in detail with reference to the accompanying drawings. In describing an embodiment, technical details that are well known in the art to which the inventive concept pertains and are not directly related to the inventive concept will be omitted. This is to avoid obscuring the essence of the inventive concept and to convey it more clearly by omitting unnecessary explanations. For the same reason, some components in the attached drawings are exaggerated, omitted, or shown schematically. Furthermore, the size of each component does not necessarily reflect its actual size. In this specification, the same reference numerals throughout the specification may refer to the same or corresponding components.

In this specification, the term ‘interaction’ may refer to a non-covalent interaction between a compound and a protein, specifically a non-covalent interaction between elements of the compound and residues of the protein.

In this specification, the term ‘binding affinity’ may refer to the strength of the binding interaction between a compound and a protein.

In this specification, the term ‘knowledge distillation’ may refer to a method by which a teacher model transfers learned knowledge to a student model. In this specification, the teacher model may be a first Artificial intelligence (AI) model 100, and the student model may be a second AI model.

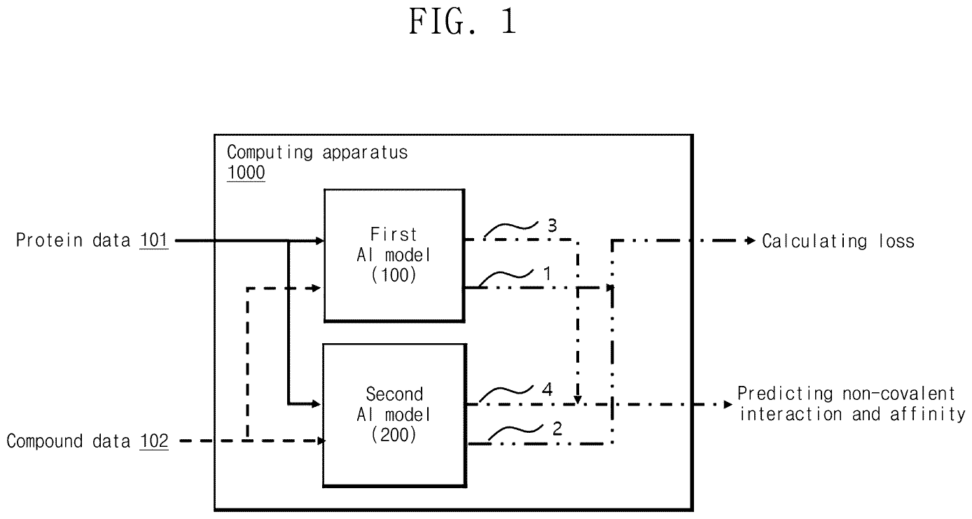

FIG. 1 is a functional block diagram illustrating an internal configuration of a computing apparatus 1000, according to an embodiment. Referring to FIG. 1, the computing apparatus 1000 may refer to any AI device that receives protein data 101 and compound data 102 to predict the binding affinity and the non-covalent binding of the corresponding protein-compound. The computing apparatus 1000 may include a first AI model 100 and a second AI model 200, and the first AI model 100 and the second AI model 200 may receive the protein data 101 and the compound data 102.

The first AI model 100 and the second AI model 200 are described as physically separate components from each other. However, this is only an example. In another embodiment, the first AI model 100 and the second AI model 200 may be logically separate structures, in which case they may be implemented by separate functions on a single server.

The first AI model 100 may be a device learned to predict binding affinity and non-covalent interactions of compound-protein by using protein data and compound data. The first AI model 100 may perform knowledge distillation by delivering an output value to the second AI model 200.

The second AI model 200 may predict the binding affinity and the non-covalent interaction of compound-protein based on the protein data, the compound data, and data received from the first AI model 100.

A loss function may be calculated based on a first output value (a long dashed-dotted-dotted line 1) of the first AI model 100 and the first output value (a long dashed-dotted-dotted line 2) of the second AI model 200. Moreover, the non-covalent interaction and the binding affinity may be predicted based on a second output value (a dashed-dotted line 3) of the first AI model 100 and a second output value (a dashed-dotted line 4) of the second AI model 200. The method for predicting the binding affinity and the non-covalent interaction of compound-protein by using the first AI model 100 and the second AI model 200 of the computing apparatus 1000 will be described in detail below.

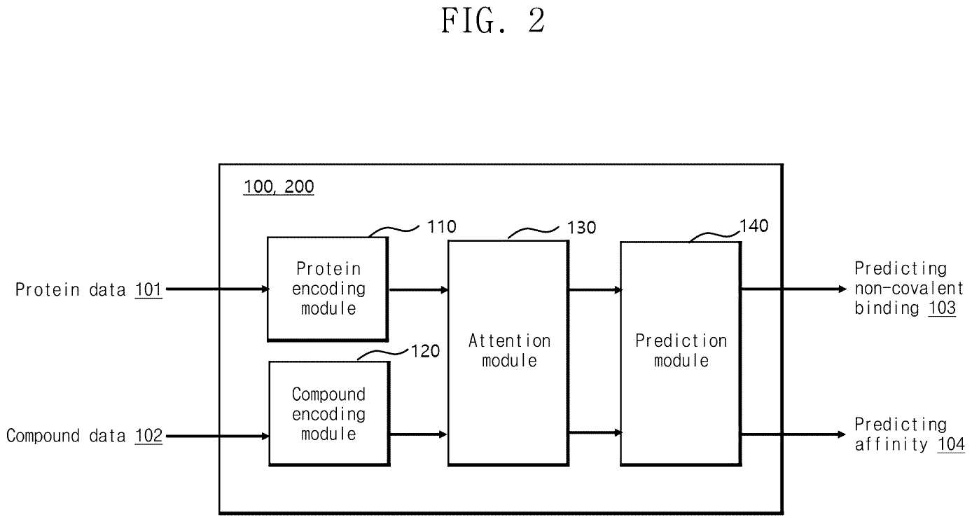

FIG. 2 is a functional block diagram showing an internal configuration of an AI model, according to an embodiment.

According to an embodiment, both the first AI model 100 and the second AI model 200 may include a protein encoding module 110, a compound encoding module 120, an attention module 130, and a prediction module 140. Hereinafter, the descriptions of the protein encoding module 110, the compound encoding module 120, the attention module 130, and the prediction module 140 may be applied to both the first AI model 100 and the second AI model 200.

The first AI model 100 and the second AI model 200 may receive the protein data 101 and the compound data 102.

The first AI model 100 may obtain the protein data 101 and the compound data 102 from a three-dimensional (3D) structure of a compound-protein complex, for example, from a PDBbind database.

The second AI model 200 may obtain the protein data 101 and the compound data 102 from a free compound-protein complex model, for example, from a BindingDB database.

Here, the protein data 101 may include a ligand of a pocket region, which is a recessed region on the protein surface, and the compound data 102 may include a target molecule region, a region that binds to the protein.

When the protein data 101 and the compound data 102 are received, the protein encoding module 110 and the compound encoding module 120 may perform encoding to use the protein data 101 and the compound data 102 as an input of the attention module 130. Here, the encoding may refer to a method of converting original data into a different format.

The protein encoding module 110 and the compound encoding module 120 may respectively generate a protein attribute vector and a compound attribute vector based on the protein data 101 and the compound data 102, and may transmit the protein attribute vector and the compound attribute vector to the attention module 130. In detail, the protein encoding module 110 and the compound encoding module 120 may obtain residue information of a protein pocket and element information of a compound by performing embedding based on the protein data 101 and the compound data 102.

Here, the “embedding” may refer to converting a sequence (e.g., an amino acid sequence or an element of a compound) into a vector having a specific numerical value to quantify data.

When receiving a protein attribute vector and a compound attribute vector, the attention module 130 may calculate an attention value based on the protein attribute vector and the compound attribute vector. Here, the attention value may represent the interaction between the compound and the protein. When receiving the protein attribute vector, the attention module 130 may calculate a protein attention value and transmit the protein attention value to the prediction module 140. When receiving the compound attribute vector, the attention module 130 may calculate a compound attention value and transmit the compound attention value to the prediction module 140.

When receiving the protein attention value and the compound attention value, the prediction module 140 may generate an interaction matrix based on the protein attention value and the compound attention value. In detail, the prediction module 140 of the first AI model 100 may generate a first interaction matrix, and the prediction module 140 of the second AI model 200 may generate a second interaction matrix. Here, the interaction matrix may be a matrix representing an interaction score between a residue of a protein and an element of a compound.

Referring to FIGS. 1 and 2, the first AI model 100 may perform learning to predict the binding affinity and the non-covalent interaction of a compound-protein complex by using the first interaction matrix as learning data. The first AI model 100 may receive the compound-protein complex and may transmit the corresponding a binding affinity prediction value and a non-covalent interaction prediction value of the compound-protein complex to the second AI model 200.

The second AI model 200 may learn the binding affinity and the non-covalent interaction of the compound-protein complex based on the output value of the first AI model and the second interaction matrix.

In this case, the second AI model 200 may be based on complex free, but the second AI model 200 may accurately predict the binding affinity of compound-protein by receiving knowledge distillation on complex-based data from the first AI model 100.

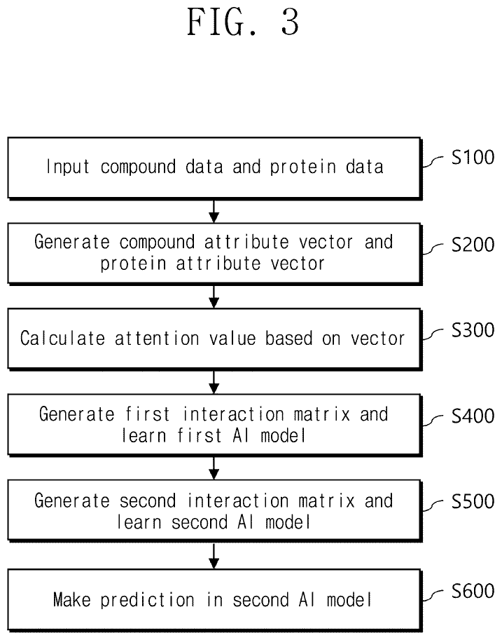

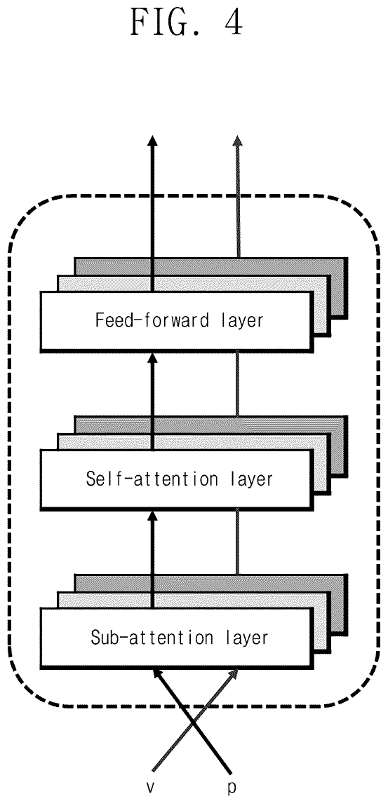

The specific details of a compound-protein binding affinity prediction method according to an embodiment will be described in detail in FIGS. 3 and 4 below.

FIG. 3 is a flowchart of a compound-protein binding affinity prediction method, according to an embodiment. Referring to FIG. 3, the binding affinity prediction method may include an operation of inputting compound data and protein data (S100), an operation of generating a compound attribute vector and a protein attribute vector (S200), an operation of calculating an attention value based on a vector (S300), an operation of generating a first interaction matrix and learning a first AI model (S400), an operation of generating a second interaction matrix and learning a second AI model (S500), and an operation of making a prediction in the second AI model (S600). Hereinafter, operations S100 to S300 may be performed by both the first AI model 100 and the second AI model 200.

Operation S100 may refer to a step in which the encoding modules 110 and 120 of the AI models 100 and 200 receive compound data and protein data. Here, the compound data and the protein data may be provided as compound-protein complex data in the first AI model 100 and may be provided as compound-protein complex free data in the second AI model 200.

The received protein data may include amino acid sequences of a residues in a pocket region that bind to a compound. Here, the second AI model 200 may use a protein binding pocket prediction tool to extract the input amino acid sequences associated with a binding pocket of the protein by receiving the compound-protein complex free data. Afterwards, the second AI model 200 may extract residue amino acid embedding of the pocket region by using another pre-learned AI model.

In an embodiment, an operation of receiving the compound data may include an operation of generating a compound graph. The compound graph may be generated using a known AI graph. Here, the compound structure graph may have atomic attributes as nodes and bond attributes as edges.

In this case, the compound data may include an elemental attribute and a bond attribute. For example, the element attribute may include an element's type, chirality, formal charge, the number of bonded hydrogen atoms, the number of bonded free electrons, hybridization, aromaticity, cyclicity, or the like. Here, the bond may refer to a bond between elements of a compound. For example, the bond attribute may include the type of bond (a single bond, a double bond, or a triple bond) and the nature of bond (directionality, chirality, stereo, or conjugation).

Operation S200 may refer to a step for generating a compound attribute vector and a protein attribute vector in the encoding modules 110 and 120 of the AI models 100 and 200.

In detail, the protein encoding module 110 may extract the protein attribute vector by using the amino acid embedding of a residue as an input. For example, the protein attribute vector may be extracted by using the amino acid embedding of the residue as an input based on Equation 1 below.

$${p}_{j} = {SelfAttLayer}_{P \to P} ( {x}_{j} , \{ {x}_{1} , … , {x}_{{L}_{p}} \} ) \qquad [ Equation 1 ]$$

Here, Pⱼ may denote the extracted protein attribute vector, and Xᵢ may denote the amino acid of a residue.

The compound encoding module 120 may extract a compound attribute vector by using the node and edge of a compound structure graph as inputs. For example, the compound attribute vector may be extracted by using the node and edge of a compound structure graph as inputs based on Equation 2 below.

$${h}_{v}^{( l )} = COMBINE ( {h}_{v}^{( l - 1 )} , AGGREGATE ( \{ ( {h}_{v}^{( l - 1 )} , {h}_{u}^{( l - 1 )} , {e}_{uv} ) : u ∈ 𝒩 ( v ) \} ) ) \qquad [ Equation 2 ]$$

Here, (v) may denote all neighbors of node v; eᵤᵥ may denote the edge between nodes u and v; and, hᵥ^{(l-1)} may denote the (l-1)-th layer of node v.

This layer may be repeatedly applied to update nodes, and the updated nodes may be used as the compound attribute vector.

Operation S300 may be a step in which the attention module 130 of the AI models 100 and 200 calculates an attention value based on the compound attribute vector and the protein attribute vector.

In particular, the attention module 130 may calculate each key, each value, and each query based on the compound attribute vector and the protein attribute vector. Here, each key, each value, and each query may be calculated by using a known attention algorithm.

The attention module 130 may include a cross-attention layer to perform cross-attention. Accordingly, the attention module 130 may cross the calculated compound query with the calculated protein query, or cross the key and value of a compound with the key and value of a protein, and may provide the crossed result to a sub-attention layer.

The sub-attention layer may perform attention by using a multi-head self-attention layer and may provide an output value to a self-attention layer. The self-attention layer may perform self-attention to provide an output value to a feed-forward layer. The feed-forward layer may perform a feed-forward operation to provide a compound attention value and a protein attention value. Accordingly, when the attention module 130 receives a compound attribute vector, the output value may be the compound attention value. When the attention module 130 receives a protein attribute vector, the output value may be the protein attention value.

FIG. 4 is a diagram illustrating an operation of calculating an attention value by using a layer, according to an embodiment. Referring to FIG. 4, the attention module 130 may cross a query of a compound attribute vector ‘v’ with the query of a protein attribute vector ‘p’, or may cross the key and value of the compound attribute vector ‘v’ with the key and value of the protein attribute vector ‘p’. The attention module 130 may include a sub-attention layer, a self-attention layer, and a feed-forward layer to calculate a compound attention value and a protein attention value by using multi-head attention, self-attention, and feed-forward. The finally calculated compound attention value may be represented as v̅ᵢ, and the finally calculated protein attention value may be represented as p̅ⱼ.

Operation S400 may be a step in which the prediction module 140 of the first AI model 100 generates a first interaction matrix and learns a first AI model. Here, the first interaction matrix may represent an interaction score between the element of a compound and the residue of a protein. The interaction score may be calculated based on Equation 3 below.

$${M}_{ij} = σ ( {\overline{v}}_{i} {W}_{1} ) \cdot {σ ( {\overline{p}}_{j} {W}_{2} )}^{T} \qquad [ Equation 3 ]$$

Here, Mᵢⱼ represents the interaction score between the i-th element and the j-th residue, and W₁ and W₂ represent learning parameters, respectively.

The prediction module 140 of the first AI model 100 may extract a latent variable based on the generated interaction score.

$${V}_{inter} = {\sum }_{i = 1}^{{N}_{a}} {\sum }_{j = 1}^{{L}_{p}} {M}_{ij} [ σ ( {\overline{v}}_{i} {W}_{3} ) \cdot σ ( {\overline{p}}_{j} {W}_{4} ) ] \qquad [ Equation 4 ]$$

Here, Vᵢₙₜₑᵣ represents the latent variable, and W₃ and W₄ represent learning parameters, respectively.

The prediction module 140 of the first AI model 100 may provide the latent variable to a fully connected layer to predict a binding affinity.

Here, a loss function of the first AI model 100 may be expressed based on Equation 5 below.

$${ℒ}_{total} = {ℒ}_{aff} + {α}_{T} {ℒ}_{inter} \qquad [ Equation 5 ]$$

Here, α_{T} denotes a weight parameter, and [symbol] _{aff} and [symbol] ᵢₙₜₑᵣ may be expressed by Equations 6 and 7, respectively.

$${ℒ}_{aff} = \frac{1}{N} {\sum }_{n = 1}^{N} {‖ {P}_{gt} - {P}_{pred} ‖}_{n}^{2} \qquad [ Equation 6 ]$$

$${ℒ}_{inter} = \frac{1}{N} {\sum }_{n = 1}^{N} {\sum }_{i = 1}^{{N}_{a}} {\sum }_{j = 1}^{{L}_{p}} - {‖ {B}_{ij} log {M}_{ij} + ( 1 - {β}_{ij} ) log ( 1 - {M}_{ij} ) ‖}_{n} \qquad [ Equation 7 ]$$

Here, P_{gt} may denote an actual binding affinity; P_{pred} may denote a predicted binding affinity; and Bᵢⱼ may denote a binary label between an actual i-th element and a j-th residue.

The first AI model 100 may deliver the output value of the first AI model to the second AI model 200. Here, the output value of the first AI model may include the predicted binding affinity, the attribute vector of a compound, the attribute vector of a protein, and a first interaction matrix.

Operation S500 may be a step in which the prediction module 140 of the second AI model 200 generates a second interaction matrix and learns a second AI model. In detail, operation S500 may refer to a step of inputting the output value of the first AI model into the second AI model to perform knowledge distillation, and may be a step in which the prediction module 140 of the second AI model 200 generates the second interaction matrix and learns the second AI model based on the output value of the first AI model. Here, the output value of the first AI model may include the predicted binding affinity, the attribute vector of a compound, the attribute vector of a protein, and a first interaction matrix.

The prediction module 140 of the second AI model 200 may learn the second AI model by generating the second interaction matrix by using the same procedure as operations S100 to S400, except that it receives compound-protein complex free data.

Operation S600 may refer to a step of predicting a non-covalent binding interaction and a binding affinity from the learned second AI model 200. In an embodiment, operation S600 may include an operation of comparing the first interaction matrix and the second interaction matrix to derive a loss function.

In particular, the second AI model 200 may calculate two loss functions through a two-stage optimization process. The first loss function may be expressed by Equations 8 and 9 below.

$${ℒ}_{{stage}_{1}} = {ℒ}_{inter} + {ℒ}_{hint} \qquad [ Equation 8 ]$$

$${ℒ}_{inter} = \frac{1}{N} {\sum }_{n = 1}^{N} {\sum }_{i = 1}^{{N}_{a}} {\sum }_{j = 1}^{{L}_{p}} - {‖ {\hat{M}}_{ij}^{T} log {M}_{ij}^{S} + ( 1 - {\hat{M}}_{ij}^{T} ) log ( 1 - {M}_{ij}^{S} ) ‖}_{n} \\ {ℒ}_{hint} = 0.5 ( \frac{1}{N} {\sum }_{n = 1}^{N} {Φ}_{n} {‖ {ψ}_{T}^{c} ( I ; {W}_{hint}^{c} ) - {ψ}_{S}^{c} ( I ; {W}_{guide}^{c} ) ‖}_{n}^{2} + \frac{1}{N} {Φ}_{n} {‖ {ψ}_{T}^{p} ( I ; {W}_{S}^{p} ( I ; {W}_{guide}^{p} ) ‖}_{n}^{2} ) \qquad [ Equation 9 ]$$

Here,

$${M}_{ij}^{S}$$

may denote the interaction between the i-th element and the j-th residue predicted by the second AI model 200;

$${\hat{M}}_{ij}^{T}$$

may denote the interaction between the i-th element and the j-th residue predicted by the first AI model 100; Wₕᵢₙₜ and W_{guide} may denote a parameter of a layer for calculating an attention value based on a compound attribute vector, and a parameter of a layer for calculating an attention value based on a protein attribute vector, respectively; ψ_{T} and ψ_{S} may denote deep neural functions of the first AI model 100 and the second AI model 200, respectively; and, Φₙ may denote a parameter that quantizes the confidence for the prediction of the first AI model 100.

The second loss function calculated from the second AI model 200 may be expressed based on Equation 10 below.

$${ℒ}_{{stage}_{2}} = {ℒ}_{reg} + {α}_{S} {ℒ}_{imit} \\ {ℒ}_{reg} = \frac{1}{N} {\sum }_{n = 1}^{N} {‖ {P}_{S} - {P}_{gt} ‖}_{n}^{2} \\ {ℒ}_{imit} = \frac{1}{N} {\sum }_{n = 1}^{N} {Φ}_{n} {‖ {P}_{S} - {P}_{T} ‖}_{n}^{2}$$

Here, α_{S} may denote a weight parameter controlling the final predicted affinity, and P_{S}, P_{T}, and P_{gt} may denote the prediction of the second AI model 200, the prediction of the first AI model 100, and an actual label, respectively.

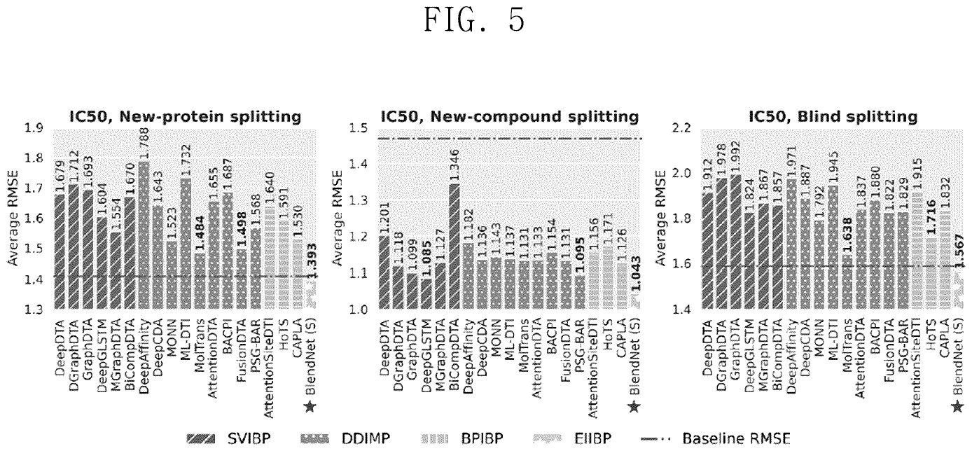

FIG. 5 is a graph for comparing the performance of models predicting a binding affinity of compound-protein, according to an embodiment. In FIG. 5, Blendnet(s) represents the second AI model 200 according to an embodiment of the inventive concept. Referring to FIG. 5, compared to other prediction models, the prediction method according to an embodiment of the inventive concept demonstrates superior prediction performance in all of new protein-based segmentation, new compound-based segmentation, and random segmentation model evaluation.

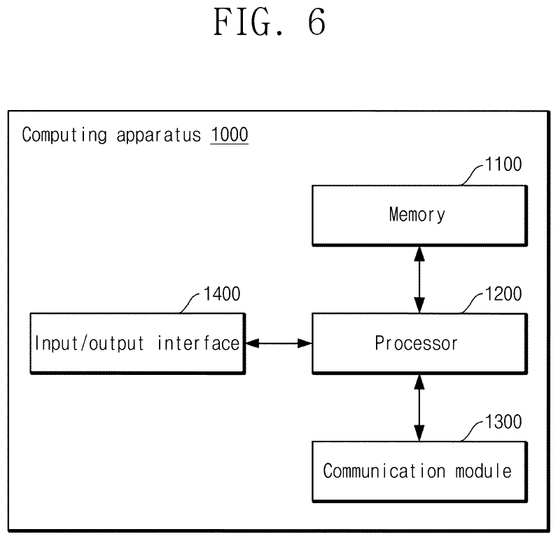

FIG. 6 is a block diagram showing a hardware configuration of a computing apparatus, according to an embodiment.

The computing apparatus 1000 may include a memory 1100, a processor 1200, a communication module 1300, and an input/output interface 1400. As shown in FIG. 6, the computing apparatus 1000 may be configured to exchange information and/or data over a network by using the communication module 1300.

The memory 1100 may include any computer-readable recording medium. According to an embodiment, the memory 1100 may include a permanent mass storage device such as a random access memory (RAM), a read only memory (ROM), a disk drive, a solid state drive (SSD), a flash memory, or the like. For another example, the permanent mass storage device such as a ROM, a SSD, a flash memory, or a disk drive may be included in the computing apparatus 1000 as a permanent storage device separate from the memory. Moreover, the memory 1100 may store the first AI model 100 and the second AI model 200, and may store an operating system and at least one program code.

These software components may be loaded from a computer-readable recording medium independent of the memory 1100. Such the separate computer-readable recording medium may include a recording medium capable of being directly connected to the computing apparatus 1000, and may include, for example, a computer-readable recording medium such as a floppy drive, a disk, a tape, a DVD/CD-ROM drive, and a memory card. For another example, the software components may be loaded into the memory 1100 through the communication module 1300, not the computer-readable recording medium. For example, at least one program may be loaded into the memory 1100 based on a computer program installed by files provided by developers or a file distribution system, which distributes a file for installing an application, through the communication module 1300.

The processor 1200 may be configured to process instructions of a computer program by performing basic arithmetic, logic, and input and output operations. The instructions may be provided to another user terminal (not shown) or another external system by the memory 1100 or the communication module 1300.

The communication module 1300 may provide a configuration or function that allows a user terminal (not shown) and the computing apparatus 1000 to communicate with each other over the network. The computing apparatus 1000 may provide a configuration or function for communicating with an external system (e.g., a separate cloud system, etc.). For example, control signals, commands, data, or the like provided under the control of the processor 1200 of the computing apparatus 1000 may be transmitted to a user terminal and/or the external system through the communication module of the user terminal and/or the external system via the communication module 1300 and a network.

Moreover, the input/output interface 1400 of the computing apparatus 1000 may be a means for interfacing with an apparatus (not shown) for an input or an output, which is connected to the computing apparatus 1000 or is included in the computing apparatus 1000. In FIG. 6, the input/output interface 1400 is shown as an element configured separately from the processor 1200, but is not limited thereto. For example, the input/output interface 1400 may be configured to be included in the processor 1200. The computing apparatus 1000 may include more components than those of FIG. 6. However, there is no need to clearly illustrate most conventional components.

The processor 1200 of the computing apparatus 1000 may be configured to manage, process, and/or store information and/or data received from a plurality of user terminals and/or a plurality of external systems.

The above-described method and/or various embodiments may be implemented by digital electronic circuits, computer hardware, firmware, software, and/or a combination thereof. Various embodiments of the inventive concept may be implemented as a data processing apparatus, for example, one or more programmable processors and/or one or more computing apparatuses, or as a computer-readable recording medium and/or a computer program stored on the computer-readable recording medium. The computer program described above may be written in any programming language, including a compiled or interpreted language, and may be distributed in any form, such as a standalone program, module, subroutine, or the like. The computer program may be distributed through a single computing apparatus, a plurality of computing apparatuses connected through the same network, and/or a plurality of computing apparatuses distributed to be connected through a plurality of different networks.

Meanwhile, embodiments disclosed in the specification may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium. The computer-readable recording medium includes all kinds of recording media in which instructions capable of being decoded by a computer are stored. For example, there may be a ROM, a RAM, a magnetic tape, a magnetic disk, a flash memory, an optical data storage device, or the like.

The above description refers to detailed embodiments for implementing the inventive concept. The inventive concept may include embodiments in which a design is changed simply or which are easily changed, as well as the embodiments described above. In addition, the inventive concept may include technologies that are easily changed and implemented by using the above-described embodiments. While the inventive concept has been described with reference to embodiments thereof, it will be apparent to those of ordinary skill in the art that various changes and modifications may be made thereto without departing from the spirit and scope of the inventive concept as set forth in the following claims

According to an embodiment of the inventive concept, compound-protein binding affinity may be accurately and effectively predicted based on compound-protein complex free.

Effects according to the inventive concept are not limited to the effects mentioned above, and other effects not mentioned will be clearly understood by those skilled in the art from the following description.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A compound-protein binding affinity prediction method performed by at least one processor, the method comprising:
receiving compound data and protein data, which interact with each other;
generating an attribute vector of a compound and an attribute vector of a protein based on the input compound data and the input protein data;
calculating an attention value based on the attribute vector of the compound and the attribute vector of the protein;
generating a first interaction matrix based on the attention value;
learning a first AI model to predict a binding affinity and a non-covalent interaction of compound-protein by using the first interaction matrix as learning data; and
predicting the binding affinity and the non-covalent interaction of the compound-protein based on an output value of the first AI model.

2. The method of claim 1, wherein the receiving of the compound data includes:
generating a compound structure graph, and
wherein the compound structure graph has an attribute of an atom as a node and has an attribute of a bond as an edge.

3. The method of claim 1, wherein the calculating of the attention value based on the attribute vector of the compound and the attribute vector of the protein includes:
calculating a key, a value, and a query based on the attribute vector of the compound and the attribute vector of the protein; and
crossing the calculated query of the compound and the calculated query of the protein, or crossing a key and a value of the compound and a key and a value of the protein to provide the crossed result to a sub-attention layer.

4. The method of claim 3, wherein the calculating of the attention value based on the attribute vector of the compound and the attribute vector of the protein further includes:
providing an output value of the sub-attention layer to a self-attention layer; and
providing an output value of the self-attention layer to a feed-forward layer.

5. The method of claim 1, wherein the learning of the first AI model includes:
calculating an interaction score between one or more elements of the compound and one or more residues of the protein;
extracting a latent variable from the score; and
providing the latent variable to a fully connected layer.

6. The method of claim 1, wherein the output value of the first AI model includes the attribute vector of the compound, the attribute vector of the protein, the first interaction matrix, and the binding affinity predicted by the first AI model.

7. The method of claim 1, wherein the predicting of the binding affinity and the non-covalent interaction of the compound-protein based on the output value of the first AI model includes:
performing knowledge distillation by inputting the output value of the first AI model into a second AI model; and
predicting the non-covalent interaction and the binding affinity in the second AI model, and
wherein data received by the first AI model is based on a compound-protein complex.

8. The method of claim 7, wherein the performing of the knowledge distillation includes:
calculating a loss function by comparing the first interaction matrix with a second interaction matrix generated by the second AI model.

9. A non-transitory computer readable recording medium including computer program to perform the compound-protein binding affinity prediction of claim 1.

10. A computing apparatus comprising:
a communication module;
a memory; and
at least one processor connected to the memory and configured to execute at least one computer-readable program included in the memory,
wherein the at least one program includes instructions for:
receiving a structure of a compound and a structure of a protein, which interact with each other;
generating an attribute vector of a compound and an attribute vector of a protein based on the input structure of the compound and the input structure of the protein;
calculating an attention value based on the attribute vector of the compound and the attribute vector of the protein;
generating a first interaction matrix based on the attention value;
learning a first AI model to predict a binding affinity and a non-covalent interaction of compound-protein by using the first interaction matrix as learning data; and
predicting the binding affinity and the non-covalent interaction of the compound-protein based on an output value of the first AI model.
